(19) United States
(12) Patent Application Publication
(10) Pub. No.: US 2026/0069121 A1
(43) Pub. Date: Mar. 12, 2026

(54) FRAME IMAGE EXTRACTION METHOD

(71) Applicant: Evident Corporation, Nagano (JP)

(72) Inventor: Shogo USUI, Nagano (JP)

(73) Assignee: Evident Corporation, Nagano (JP)

(21) Appl. No.: 19/392,626

(22) Filed: Nov. 18, 2025

Related U.S. Application Data
(62) Division of application No. 18/136,548, filed on Apr. 19, 2023.

(30) Foreign Application Priority Data
May 20, 2022 (JP) 2022-083101

(51) Int. Cl. A61B 1/05; G06V 10/25; G16H 30/20; G16H 30/40

(52) U.S. Cl. CPC A61B 1/05; G06V 10/25; G16H 30/20; G16H 30/40

(57) ABSTRACT

A frame image extraction method including: during recording processing of a moving image including a plurality of frame images generated on a basis of an imaging signal output from an imaging element included in an insertion portion inserted into a test subject, among the plurality of frame images, adding, as a tag, information regarding an operation to a frame image corresponding to a timing when an operation receiving unit receives the operation, and adding, as a tag, information regarding a specific feature image to a frame image recognized as the specific feature image; and extracting a frame image from among the plurality of frame images included in the moving image on a basis of at least one type of tag among two or more types of tags selected from among a plurality of types of the tags.

### CROSS REFERENCE TO RELATED APPLICATIONS

This application is a divisional application of U.S. Patent Application No. 18/136,548, filed on April 19, 2023, which is based upon and claims the benefit of priority of the prior Japanese Patent Application No. 2022-083101, filed May 20, 2022, the entire contents of each of which are incorporated herein by reference.

### BACKGROUND OF THE INVENTION

### Field of the Invention

The disclosure of the present specification relates to a frame image extraction method.

### Description of the Related Art

In endoscopic inspection, there is a work of capturing a moving image of the inside of a test subject and checking after the inspection. This work is not easy as the captured moving image becomes a long time. Therefore, there is a demand for a technique capable of efficiently checking only an important portion (an important frame image included in a moving image) in a captured moving image.

JP 5568196 B1 describes an image processing device that processes an image acquired in an examination using a capsule endoscope. The image processing device compares imaging times of a plurality of types of feature images (for example, a pyloric image, a clip image, a Vater's papilla image, and a Bauhin valve image) extracted from an image group (current image group) acquired in the examination with imaging times of a plurality of types of corresponding feature images extracted from an image group (previous image group) acquired in the previous examination with respect to the same test subject, and adds, to an image imaged within a time interval between the feature images of the current image group, a flag indicating that it is an observation attention image in a case where a time interval between the feature images of the current image group is longer than a time interval between the corresponding feature images of the previous image group by a reference value or more. Then, at the time of displaying the current image group, the image to which the flag is added is displayed in a format different from that of another image in order to attract the attention of a medical personnel and allow the medical personnel to perform intensive observation.

### SUMMARY OF THE INVENTION

An endoscopic device according to an aspect of the present invention includes: an insertion portion that is inserted into a test subject and includes an imaging element; an operation receiving unit that receives an operation; and a processor, in which the processor, during recording processing of a moving image including a plurality of frame images generated on a basis of an imaging signal output from the imaging element, among the plurality of frame images, adds, as a tag, information regarding an operation to a frame image corresponding to a timing when the operation receiving unit receives the operation, and adds, as a tag, information regarding a specific feature image to a frame image recognized as the specific feature image, and extracts a frame image from among the plurality of frame images included in the moving image on a basis of at least one type of tag among two or more types of tags selected from among a plurality of types of the tags.

A frame image extraction method according to an aspect of the present invention includes: during recording processing of a moving image including a plurality of frame images generated on a basis of an imaging signal output from an imaging element included in an insertion portion inserted into a test subject, among the plurality of frame images, adding, as a tag, information regarding an operation to a frame image corresponding to a timing when an operation receiving unit receives the operation, and adding, as a tag, information regarding a specific feature image to a frame image recognized as the specific feature image; and extracting a frame image from among the plurality of frame images included in the moving image on a basis of at least one type of tag among two or more types of tags selected from among a plurality of types of the tags.

A computer-readable medium according to an aspect of the present invention is a non-transitory computer-readable medium storing a program for causing a processor to execute processing of: during recording processing of a moving image including a plurality of frame images generated on a basis of an imaging signal output from an imaging element included in an insertion portion inserted into a test subject, among the plurality of frame images, adding, as a tag, information regarding an operation to a frame image corresponding to a timing when an operation receiving unit receives the operation, and adding, as a tag, information regarding a specific feature image to a frame image recognized as the specific feature image; and extracting a frame image from among the plurality of frame images included in the moving image on a basis of at least one type of tag among two or more types of tags selected from among a plurality of types of the tags.

An endoscopic system according to an aspect of the present invention includes: an endoscopic device; and a control device, in which the endoscopic device includes: an insertion portion that is inserted into a test subject and includes an imaging element, and an operation receiving unit that receives an operation, and the control device, during recording processing of a moving image including a plurality of frame images generated on a basis of an imaging signal output from the imaging element, among the plurality of frame images, adds, as a tag, information regarding an operation to a frame image corresponding to a timing when the operation receiving unit receives the operation, and adds, as a tag, information regarding a specific feature image to a frame image recognized as the specific feature image, and extracts a frame image from among the plurality of frame images included in the moving image on a basis of at least one type of tag among two or more types of tags selected from among a plurality of types of the tags.

### BRIEF DESCRIPTION OF THE DRAWINGS

FIG. 1 is a diagram illustrating an external configuration of an endoscopic device according to an embodiment;

FIG. 2 is a diagram illustrating an internal configuration of the endoscopic device according to an embodiment;

FIG. 3 is a flowchart illustrating a flow of processing performed by a control unit in response to a moving image recording start operation;

FIG. 4 is a flowchart illustrating a flow of processing performed by the control unit in response to a moving image selection operation;

FIG. 5 is a diagram illustrating a condition selection screen displayed in S220 and including a tag selected in S230;

FIG. 6 is a diagram describing a specific example of extraction in S240;

FIG. 7 is a diagram illustrating a modification of the condition selection screen displayed in S220;

FIG. 8 is a diagram illustrating a modification of the condition selection screen displayed in S220;

FIG. 9 is a diagram illustrating an example of selecting a tag by region designation; and

FIG. 10 is a diagram illustrating a hardware configuration of a computer.

### DESCRIPTION OF THE EMBODIMENTS

In endoscopic inspection in industrial field, internal (for example, a blade inside a turbine or the like) features of a test subject are similar. Therefore, in a case where an image is extracted on the basis of only features of an image as in the image processing device described in JP 5568196 B1, narrowing of important images becomes insufficient. In addition, in a case where a device automatically extracts an image as in the image processing device described in JP 5568196 B1, a user (inspector) cannot know on what basis the image is extracted.

An embodiment of the present invention will be described below with reference to the drawings.

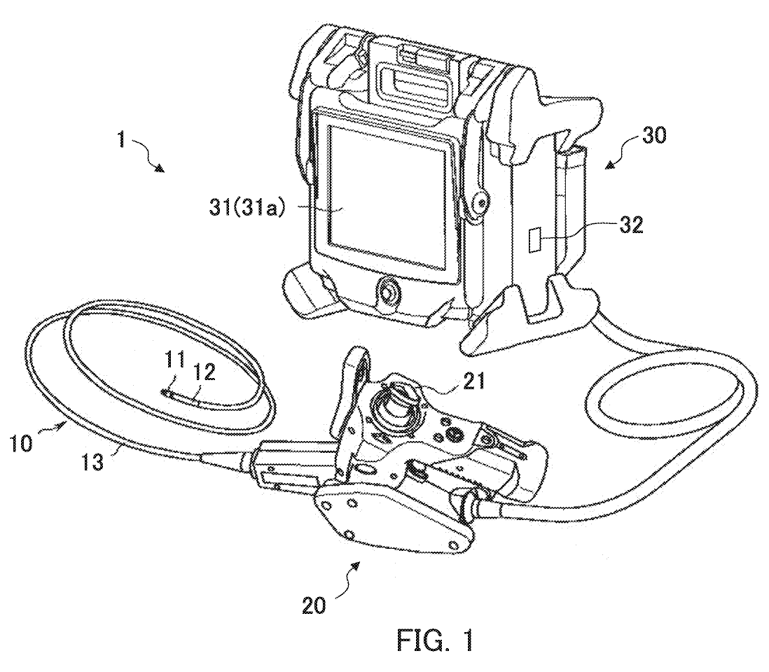

FIG. 1 is a diagram illustrating an external configuration of an endoscopic device 1 according to an embodiment. The endoscopic device 1 illustrated in FIG. 1 is used in endoscopic inspection in industrial field, and includes an insertion portion 10, an operation portion 20, and a main body portion 30.

The insertion portion 10 has an elongated shape that can be inserted into a test subject such as a turbine or an engine, and includes a distal end portion 11, a bending portion 12 formed to be bendable, and an elongated flexible tube portion 13 having flexibility.

The operation portion 20 includes a joystick (bending operator) 21 that receives an operation (bending operation) for bending the bending portion 12 in a desired direction, a plurality of buttons (not illustrated) that receives an operation for performing various inputs, and the like.

The main body portion 30 includes a display unit 31, an external interface 32, and the like. The display unit 31 is a display device such as a liquid crystal display (LCD), and performs display of an image inside the test subject, which is a subject, various screens such as a condition selection screen, reproduction display of a recorded moving image, and the like. In addition, the display unit 31 includes a touch panel 31a that receives a touch operation for performing various inputs. The touch panel 31a and the operation portion 20 described above are an example of an operation receiving unit that receives an operation of the user (inspector). An external device such as an external storage device (for example, a universal serial bus (USB) memory) is connected to the external interface 32.

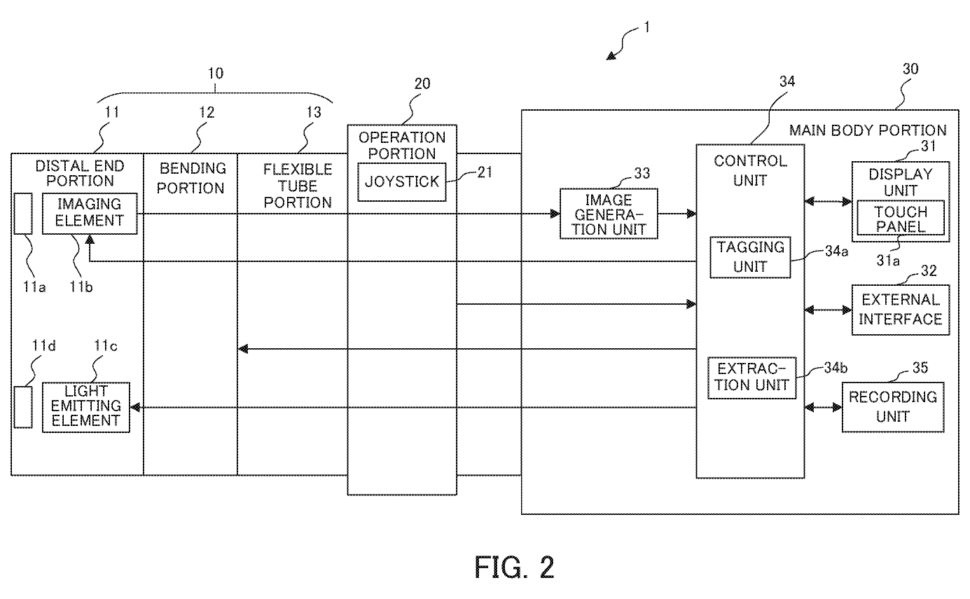

FIG. 2 is a diagram illustrating an internal configuration of the endoscopic device 1 according to an embodiment. As illustrated in FIG. 2, in the endoscopic device 1, the distal end portion 11 of the insertion portion 10 includes an imaging optical system 11a, an imaging element 11b, a light emitting element 11c, and an illumination optical system 11d.

The imaging optical system 11a forms a subject image on the imaging element 11b. The imaging element 11b captures (photoelectrically converts) the subject image formed by the imaging optical system 11a to generate an imaging signal, and outputs the imaging signal to the main body portion 30 (image generation unit 33). The imaging element 11b is, for example, a charge coupled device (CCD) image sensor, a complementary metal oxide semiconductor (CMOS) image sensor, or the like.

The light emitting element 11c emits illumination light for illuminating the subject. The light emitting element 11c is a light emitting diode (LED) or the like. The illumination optical system 11d irradiates the subject with the illumination light emitted from the light emitting element 11c. Note that, in the present embodiment, the subject is illuminated with the illumination light emitted from the light emitting element 11c, but for example, the subject may be configured to be illuminated by guiding the illumination light emitted from a light source unit included in the main body portion 30 with a light guide inserted in the insertion portion 10 or the like.

The operation portion 20 receives an operation on the joystick 21, the plurality of buttons, which is not illustrated, and the like, and outputs a signal corresponding to the operation to the main body portion 30 (control unit 34).

The main body portion 30 includes the image generation unit 33, the control unit 34, and a recording unit 35 in addition to the display unit 31 and the external interface 32 described above. Note that the touch panel 31a included in the display unit 31 receives a touch operation and outputs a signal corresponding to the touch operation to the control unit 34.

The image generation unit 33 generates a frame image by performing predetermined signal processing with respect to the imaging signal output from the imaging element 11b, and sequentially outputs the generated frame image to the control unit 34. The image generation unit 33 includes, for example, an image generation circuit.

The control unit 34 controls each portion of the endoscopic device 1. For example, the control unit 34 controls driving of the imaging element 11b, lighting up/out of illumination (light emitting element 11c), bending of the bending portion 12, display of the display unit 31, and the like. The control of lighting up/out the illumination is performed according to an operation (illumination operation) on the operation portion 20 or the touch panel 31a, and the control of the bending of the bending portion 12 is performed according to an operation (bending operation) on the operation portion 20 (joystick 21).

In addition, the control unit 34 performs various types of processing. For example, the control unit 34 performs processing of displaying frame images sequentially output from the image generation unit 33 on the display unit 31, processing of recording a moving image including the plurality of frame images sequentially output from the image generation unit 33 on the recording unit 35 or an external storage device connected to the external interface 32 (moving image recording processing), processing of displaying a moving image recorded in the recording unit 35 or the external storage device connected to the external interface 32 on the display unit 31 (reproduction and display), and the like.

In addition, during the moving image recording processing, the control unit 34 performs, for example, feature image recognition processing of recognizing a specific feature image, measurement processing of measuring the length of a flaw or the like shown in the frame image, processing of providing a comment to the frame image, processing of providing an evaluation result to the frame image, processing of providing a marking to the frame image, and the like, in accordance with an operation (measurement operation, comment providing operation, evaluation result providing operation, marking providing operation, and the like) on the operation portion 20 or the touch panel 31a. Note that, in the processing of providing a comment or an evaluation result, an arbitrary comment or an arbitrary evaluation result may be input and provided by an operation on the operation portion 20 or the touch panel 31a, or a comment or an evaluation result selected from a plurality of comments or a plurality of evaluation results prepared in advance may be provided.

In addition, the control unit 34 includes a tagging unit 34a and an extraction unit 34b.

During the moving image recording processing, the tagging unit 34a adds, as a tag, information regarding an operation to a frame image corresponding to a timing when the operation portion 20 or the touch panel 31a receives the operation, among a plurality of frame images included in the moving image. For example, the tagging unit 34a adds, as a tag, information regarding the illumination operation of illumination lighting-up to a frame image corresponding to a timing when the illumination lighting-up operation is received, and adds, as a tag, information regarding the illumination operation of illumination lighting-out to a frame image corresponding to a timing when the illumination lighting-out operation is received. In addition, the tagging unit 34a adds, as a tag, information regarding the bending operation (including information of bending angle) to a frame image corresponding to a timing when the bending operation is received. Note that, in a case where the bending operation is continuously performed, information regarding the bending operation is added as a tag to a frame image corresponding to a timing included in a period in which the bending operation is continuously performed. In addition, the tagging unit 34a adds, as a tag, information (including information of a measurement target (for example, a flaw)) regarding the measurement operation of the measurement processing start to a frame image corresponding to a timing when the operation of starting the measurement processing is received, and adds, as a tag, information (including information of a measurement target and a measurement result (for example, a measurement value of a flaw)) regarding the measurement operation of the measurement processing end to a frame image corresponding to a timing when the operation of ending the measurement processing is received. In addition, the tagging unit 34a adds, as a tag, information regarding the comment providing operation to a frame image corresponding to a timing when the comment providing operation is received. In addition, the tagging unit 34a adds, as a tag, information regarding the evaluation result providing operation to a frame image corresponding to a timing when the evaluation result providing operation is received. In addition, the tagging unit 34a adds, as a tag, information regarding the marking providing operation to a frame image corresponding to a timing when the marking providing operation is received.

In addition, during the moving image recording processing, the tagging unit 34a adds, as a tag, information regarding a specific feature image to a frame image recognized as the specific feature image by the feature image recognition processing among a plurality of frame images included in a moving image. The specific feature image is, for example, an image representing a part of interest of the user. Specifically, it is an image representing a gear, an image representing a blade, an image representing an elbow, and the like. Such a specific feature image is determined, for example, according to a feature image selection operation on the operation portion 20 or the touch panel 31a before the moving image recording processing.

The extraction unit 34b extracts a frame image from among a plurality of frame images included in a moving image recorded by the moving image recording processing on the basis of at least one type of tag among two or more types of tags selected from among a plurality of types of tags. For example, the extraction unit 34b extracts a frame image on the basis of an AND condition or an OR condition of the two or more types of tags selected from among the plurality of types of tags. The two or more types of tags are selected, for example, according to a tag selection operation on the operation portion 20 or the touch panel 31a. The tag selection operation is an operation of selecting two or more types of tags from tags added to the frame image included in the moving image.

Such a control unit 34 is configured to include, for example, a processor such as a central processing unit (CPU), random access memory (RAM), and read only memory (ROM). Then, the processor executes a program stored in the ROM while using the RAM as a work area or the like, thereby implementing the function of the control unit 34. The control unit 34, or the control unit 34 and the image generation unit 33 may include an application specific integrated circuit (ASIC) or a field programmable gate array (FPGA).

The recording unit 35 records a moving image or the like. The recording unit 35 may record a program executed by the processor of the control unit 34. The recording unit 35 is a nonvolatile memory such as a hard disk drive (HDD) or a solid state drive (SSD).

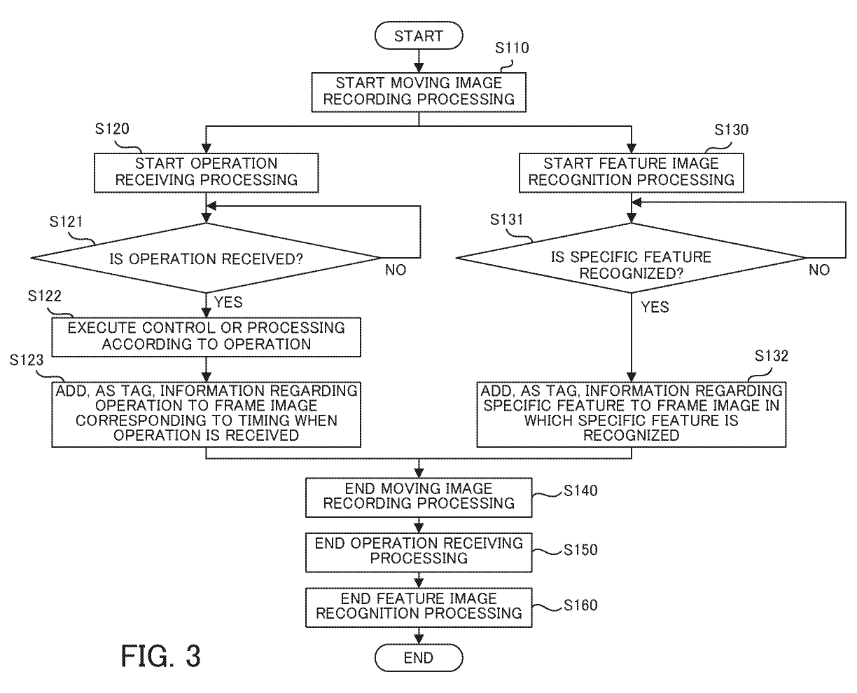

In the endoscopic device 1 configured as described above, at the time of endoscopic inspection, when the user inserts the insertion portion 10 into the test subject while checking the image displayed on the display unit 31, and performs the moving image recording start operation on the operation portion 20 or the touch panel 31a when the distal end portion 11 reaches a desired observation position, the control unit 34 starts the processing illustrated in FIG. 3.

FIG. 3 is a flowchart illustrating a flow of processing performed by the control unit 34 in response to the moving image recording start operation. In the processing illustrated in FIG. 3, first, the control unit 34 starts the moving image recording processing (S110). The moving image recording processing is processing of recording a moving image including a plurality of frame images sequentially output from the image generation unit 33 in the recording unit 35 or the external storage device connected to the external interface 32.

Next, the control unit 34 performs processing of S120 to S123 and processing of S130 to S132 in parallel.

In the processing of S120 to S123, first, the control unit 34 starts operation reception processing (S120). The operation reception processing is processing of receiving an operation on the operation portion 20 or the touch panel 31a. The processing of S121 to S123 subsequent to S120 is processing repeatedly performed until a moving image recording end operation on the operation portion 20 or the touch panel 31a is received. In the processing of S121 to S123, first, the control unit 34 determines whether an operation on the operation portion 20 or the touch panel 31a has been received (S121), and when the determination result is NO, repeats this determination. On the other hand, when the determination result in S121 is YES, the control unit 34 executes corresponding control or processing according to the received operation (S122), and the tagging unit 34a adds, as a tag, information regarding the operation to the frame image corresponding to the timing when the operation is received (S123).

In the processing of S130 to S132, first, the control unit 34 starts the feature image recognition processing (S130). The feature image recognition processing is processing of recognizing a specific feature image (more specifically, an image representing a specific feature) in frame images (also frame images included in a moving image) sequentially output from the image generation unit 33. Note that the feature image recognition processing is also processing of recognizing a specific feature in the frame images sequentially output from the image generation unit 33. The image representing the specific feature is, for example, an image representing a part of interest of the user (an image representing a gear, a blade, an elbow, or the like), and is determined according to the feature image selection operation on the operation portion 20 or the touch panel 31a before the moving image recording processing (before starting the moving image recording processing). The feature image selection operation may be, for example, an operation of selecting an image representing a specific feature from among a plurality of feature images (exemplary feature images) displayed on the display unit 31, or may be an operation of selecting an item of a specific part from among items of a plurality of parts (“gear”, “blade”, “elbow”, and the like) displayed on the display unit 31. The processing of S131 and S132 subsequent to S130 is processing repeatedly performed until the moving image recording end operation on the operation portion 20 or the touch panel 31a is received. In the processing of S131 and S132, first, the control unit 34 determines whether a specific feature (image representing the specific feature) is recognized by the feature image recognition processing (S131), and when the determination result is NO, repeats this determination. On the other hand, when the determination result in S131 is YES, the tagging unit 34a adds, as a tag, information regarding the specific feature (feature image) to the frame image in which the specific feature is recognized (the frame image recognized as the image representing the specific feature) (S132).

Then, when the operation portion 20 or the touch panel 31a receives the moving image recording end operation, the control unit 34 ends the moving image recording processing started in S110 (S140), ends the operation reception processing started in S120 (S150), ends the feature image recognition processing in S130 (S160), and ends the processing illustrated in FIG. 3. Thus, for example, the moving image including the frame image to which the tag is added is recorded in the recording unit 35 or the external storage device connected to the external interface 32.

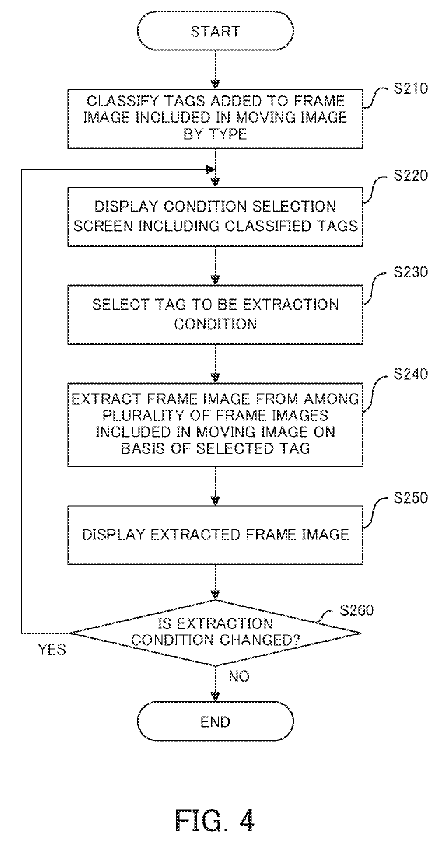

After the moving image is recorded in this manner, in the endoscopic device 1, when the user selects the moving image (the moving image including the frame image to which the tag is added) recorded in the recording unit 35 or the external storage device connected to the external interface 32 by the moving image selection operation on the operation portion 20 or the touch panel 31a in order to check the recorded moving image, the control unit 34 starts processing illustrated in FIG. 4.

FIG. 4 is a flowchart illustrating a flow of processing performed by the control unit 34 in response to the moving image selection operation. In the processing illustrated in FIG. 4, first, the control unit 34 classifies the tags added to the frame image included in the moving image selected by the moving image selection operation by type (S210). Next, the control unit 34 causes the display unit 31 to display a condition selection screen including the tags classified in S210 (S220).

Next, on the condition selection screen displayed in S220, the control unit 34 selects a tag to be an extraction condition according to the tag selection operation on the operation portion 20 or the touch panel 31a (S230). Here, in a case where a plurality of tags is selected, an AND condition or an OR condition of the plurality of tags can be an extraction condition. Specifically, in a case where the AND condition is set, a frame image to which all of the plurality of tags are added is extracted, and in a case where the OR condition is set, a frame image to which at least one of the plurality of set tags is added is extracted. In a case where the AND condition is set and the frame image is extracted, frame images more important than in the case of the OR condition can be narrowed down. On the other hand, when the OR condition is set and the frame image is extracted, in addition to an important frame image, frame images before and after the frame image is acquired can be acquired.

Next, the extraction unit 34b extracts a frame image from among the plurality of frame images included in the moving image on the basis of the tag serving as the extraction condition selected in S230 (S240). Next, the control unit 34 causes the display unit 31 to display (reproduces and displays) the frame image extracted in S240 (S250). Note that the processing of S240 and S250 is also to extract a corresponding section (corresponding period) in the moving image on the basis of the tag serving as the extraction condition selected in S230, and reproduce and display a partial moving image of the corresponding section.

Next, the control unit 34 determines whether an extraction condition change operation has been performed on the operation portion 20 or the touch panel 31a (S260), and when the determination result is YES, the processing returns to S220. On the other hand, when the determination result in S260 is NO, the control unit 34 ends the processing illustrated in FIG. 4.

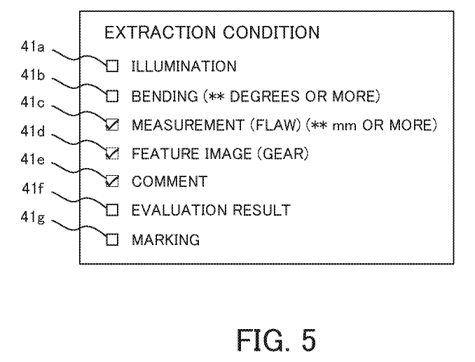

FIG. 5 is a diagram illustrating a condition selection screen displayed in S220 and including a tag selected in S230. The condition selection screen illustrated in FIG. 5 includes tags related to “illumination” (illumination operation), “bending” (bending operation), “measurement (flaw)” (flaw measurement operation), “feature image (gear)” (specific feature image (gear)), “comment” (comment providing operation), “evaluation result” (evaluation result providing operation), and “marking” (marking providing operation), as tags of types classified in S210. In addition, check boxes 41 (41a to 41g) corresponding to tags are also included. The user can select a desired tag from among them by checking the check box 41 corresponding to the desired tag. On the condition selection screen illustrated in FIG. 5, the check boxes 41c, 41d, and 41e are checked, and the tags related to “measurement (flaw)”, “feature image (gear)”, and “comment” are selected. In addition, on the condition selection screen illustrated in FIG. 5, a range of a bending angle in “bending” (for example, “** degrees or more”) and a range of a measurement value in “measurement (flaw)” (for example, “** mm or more”) can also be designated, and the range of extraction can also be limited.

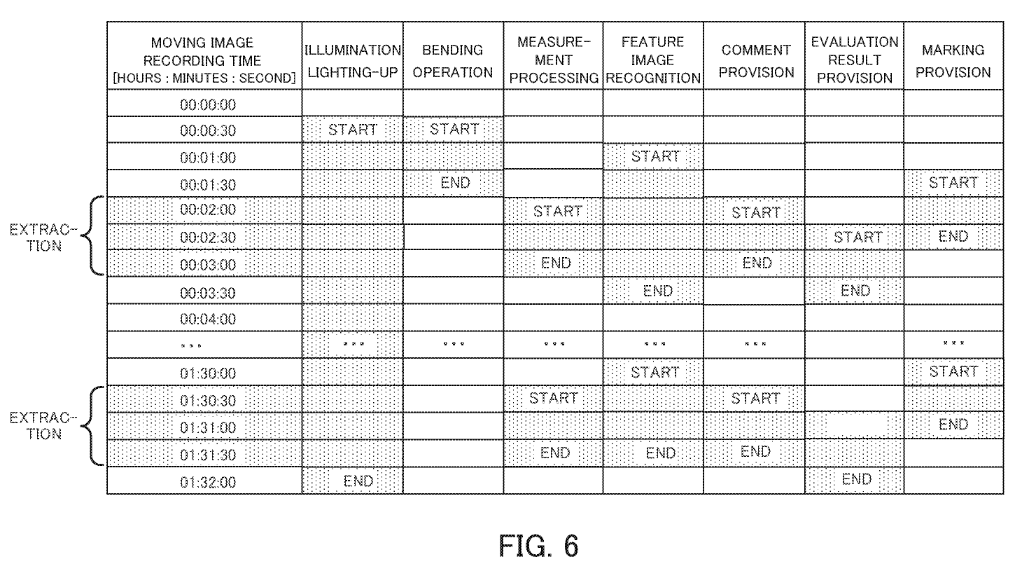

FIG. 6 is a diagram describing a specific example of extraction in S240. In this specific example, it is assumed that, on the condition selection screen illustrated in FIG. 5, the tags related to “measurement (flaw)”, “feature image (gear)”, and “comment” are selected as tags for the extraction condition, and the AND condition of these tags is set as the extraction condition. In this case, in the extraction in S240, first, the corresponding section in the moving image is specified for each selected tag. In detail, the measurement processing section is specified on the basis of the frame image to which the tag related to “measurement (flaw)” is added, the feature image recognition section is specified on the basis of the frame image to which the tag related to “feature image (gear)” is added, and the comment providing section is specified on the basis of the frame image to which the tag related to “comment” is added. The measurement processing section is a section from the frame image to which the tag related to the measurement operation of the measurement processing start is added to the frame image to which the tag related to the measurement operation of the measurement processing end is added. The feature image recognition section is a section of the frame image to which the tag related to the specific feature image (gear) is added. The comment providing section is a section of a predetermined time (for example, one minute) including the time point of the frame image to which the tag related to the comment providing operation is added. In this specific example, as illustrated in FIG. 6, in the moving image having a moving image recording time of “01:32:00”, the measurement processing section is specified as a section of “00:02:00” to “00:03:00” and a section of “01:30:30” to “01:31:30”, the feature image recognition section is specified as a section of “00:01:00” to “00:03:30” and a section of “01:30:00” to “01:31:30”, and the comment providing section is specified as a section of “00:02:00” to “00:03:00” and a section of “01:30:30” to “01:31:30”. In this way, when the sections for the selected tags are specified, next, the sections satisfying the AND condition of the sections for the tags are extracted. In this specific example, the section of “00:02:00” to “00:03:00” and the section of “01:30:30” to “01:31:30” are extracted. That is, this means that frame images included in the sections are extracted. In this specific example, the frame images included in the sections are a frame image on which the flaw measurement processing has been performed, a frame image representing the gear, and a frame image within a predetermined time (for example, one minute) including the time point when the comment is provided.

Note that, for reference, FIG. 6 also illustrates sections specified on the basis of frame images to which other (unselected) tags are added. In detail, an illumination lighting-up section specified on the basis of the frame image to which the tag related to “illumination” is added, a bending operation section specified on the basis of the frame image to which the tag related to “bending” is added, an evaluation result providing section specified on the basis of the frame image to which the tag related to “evaluation result” is added, and a marking providing section specified on the basis of the frame image to which the tag related to “marking” is added are also illustrated. The illumination lighting-up section is a section from the frame image to which the tag related to the illumination operation of illumination lighting-up is added to the frame image to which the tag related to the illumination operation of illumination lighting-out is added. The bending operation section is a section of the frame image to which the tag related to the bending operation is added. The evaluation result providing section is a section of a predetermined time (for example, one minute) including the time point of the frame image to which the tag related to the evaluation result providing operation is added. The marking providing section is a section of a predetermined time (for example, one minute) including the time point of the frame image to which the tag related to the marking providing operation is added. In FIG. 6, the illumination lighting-up section is a section of “00:00:30” to “01:32:00”, the bending operation section is a section of “00:00:30” to “00:01:30”, the evaluation result providing section is a section of “00:02:30” to “00:03:30” and a section of “01:31:00” to “01:32:00”, and the marking providing section is a section of “00:01:30” to “00:02:30” and a section of “01:30:00” to “01:31:00”.

As described above, with endoscopic device 1 according to the present embodiment, the frame image can be extracted not only on the basis of the extraction condition using one type of condition element (one type of tag) but also on the basis of the extraction condition using a plurality of types of condition elements (a plurality of types of tags) from among the plurality of frame images included in the moving image acquired by the endoscopic inspection, so that the important frame images can be more narrowed and extracted. In addition, since the user can select a condition element to be an extraction condition, the user can grasp on what basis the extraction has been performed.

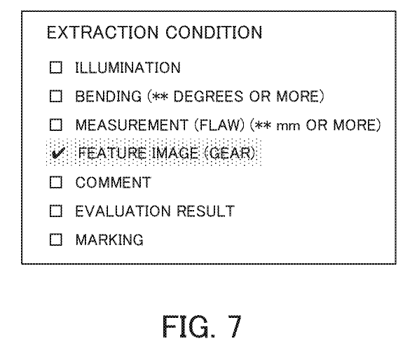

Note that, in the present embodiment, in the tags included in the condition selection screen displayed in S220, as illustrated in FIG. 7, some tags may be fixed as selected. FIG. 7 is a diagram illustrating a modification of the condition selection screen displayed in S220. In the condition selection screen illustrated in FIG. 7, the tag related to the “feature image (gear)” is fixed as selected. In addition, the tag is displayed in a format different from other tags so that it can be identified as being fixed as selected. In this example, the predetermined pattern is displayed in a format different from other tags by being superimposed, but may be displayed in a format different from other tags by being grayed out. According to the condition selection screen illustrated in FIG. 7, by selecting a tag other than the tag related to the “feature image (gear)”, the user can further narrow down the section by the selected tag in the feature image recognition section (for example, the section of “00:01:00” to “00:03:30” and the section of “01:30:00” to “01:31:30”, which are the feature image recognition sections illustrated in FIG. 6) in the moving image. Note that, in a case where the moving image includes frame images to which tags related to a plurality of types of specific feature images is added, such as a frame image to which the tag related to “feature image (gear)” is added or a frame image to which the tag related to “feature image (blade)” is added, the user may be allowed to designate a tag related to a specific feature image fixed as selected on the condition selection screen illustrated in FIG. 7 (for example, designate the tag related to the “feature image (blade)”). With such a condition selection screen illustrated in FIG. 7, it is possible to set a more important tag among a plurality of tags in advance, and thus, it is possible to more accurately extract a target frame image.

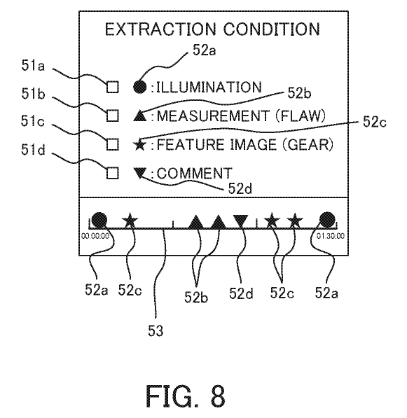

In addition, in the present embodiment, as illustrated in FIG. 8, the condition selection screen displayed in S220 may further include a time bar 53 of the moving image, and the tag classified in S210 may be identifiably displayed on the time bar 53. FIG. 8 is a diagram illustrating a modification of the condition selection screen displayed in S220. The condition selection screen illustrated in FIG. 8 includes tags related to “illumination”, “measurement (flaw)”, “feature image (gear)”, and “comment” as tags classified in S210, and includes check boxes 51 (51a to 51d) and icons 52 (52a to 52d) corresponding to the respective tags. In addition, the condition selection screen illustrated in FIG. 8 further includes the time bar 53 of the moving image, and the icons 52 corresponding to the tags classified in S210 are displayed at corresponding positions on the time bar 53. With such a condition selection screen illustrated in FIG. 8, the user can select a desired tag by checking the check box 51 of the desired tag while checking the icon 52 displayed on the time bar 53. Note that, on the time bar 53, the position where the icon 52c corresponding to the tag related to “feature image (gear)” is displayed is, for example, the position of the timing when the feature image starts to be recognized. With such a condition selection screen illustrated in FIG. 8, it is possible to visually grasp the timing when the tag is provided and the type of the tag, and it is possible to more intuitively extract the frame image.

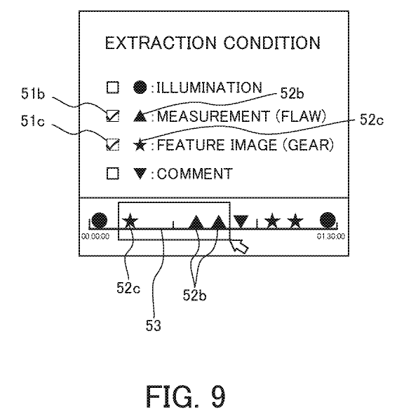

In addition, on the condition selection screen illustrated in FIG. 8, the tag may be selected by selecting the icon 52 displayed on the time bar 53 by region designation (for example, drag operation) as illustrated in FIG. 9. FIG. 9 is a diagram illustrating an example of selecting a tag by region designation. In the selection example illustrated in FIG. 9, the icon 52b corresponding to the tag related to “measurement (flaw)” and the icon 52c of the tag related to “feature image (gear)” displayed on the time bar 53 are selected by region designation, so that the tag related to “measurement (flaw)” and the tag related to “feature image (gear)” are selected. In this case, the check boxes 51b and 51c of the tags selected by the region designation are automatically checked. In this manner, the tags may be selected by region designation.

In addition, in the present embodiment, a partial moving image, which is a part of the moving image, may be generated using the frame image extracted in S240 (corresponding section of the moving image extracted in S240), and may be recorded in the recording unit 35 or the external storage device connected to the external interface 32. Thus, the partial moving image can be checked later.

In addition, in the present embodiment, the specific feature image recognized by the feature image recognition processing during the moving image recording processing may be determined, for example, on the basis of the content of a tag related to a specific feature image added by the tagging unit 34a to a frame image included in a moving image recorded by the moving image recording processing performed with respect to a test subject of the same type under the operation of an expert inspector previously. Thus, for example, even in the moving image recording processing performed under an inexperienced inspector, a tag related to a specific feature image similar to that in the moving image recording processing performed under the operation of an expert inspector can be added.

In addition, in the present embodiment, the specific feature image recognized by the feature image recognition processing during the moving image recording processing may be obtained, for example, by machine learning of the feature of the frame image to which a specific type of a tag is added, included in a moving image recorded by the moving image recording processing performed previously with respect to a test subject of the same type. Thus, for example, in a case where the specific feature image is obtained by machine learning of the feature of a frame image to which the tag related to a flaw measurement operation is added, the tag can be added to a frame image on which the flaw measurement operation is to be performed. The tag at this time may include information indicating that it is a frame image on which the flaw measurement operation is to be performed. In addition, the specific feature image at this time may be obtained by machine learning of the feature of a frame image to which the tag related to the flaw measurement operation (for example, an operation of measuring a flaw of “** mm or more”) in which the range of the measurement result (measurement value) is limited is added. Thus, the tag can be added to the frame image on which the flaw measurement operation with a limited range is to be performed.

In addition, in the present embodiment, the main body portion 30 may include a communication interface connected to a network in a wired or wireless manner and performing communication with the external device (server or the like) connected to the network. Thus, for example, data (moving image or the like) acquired by the endoscopic device 1 can be shared on the cloud.

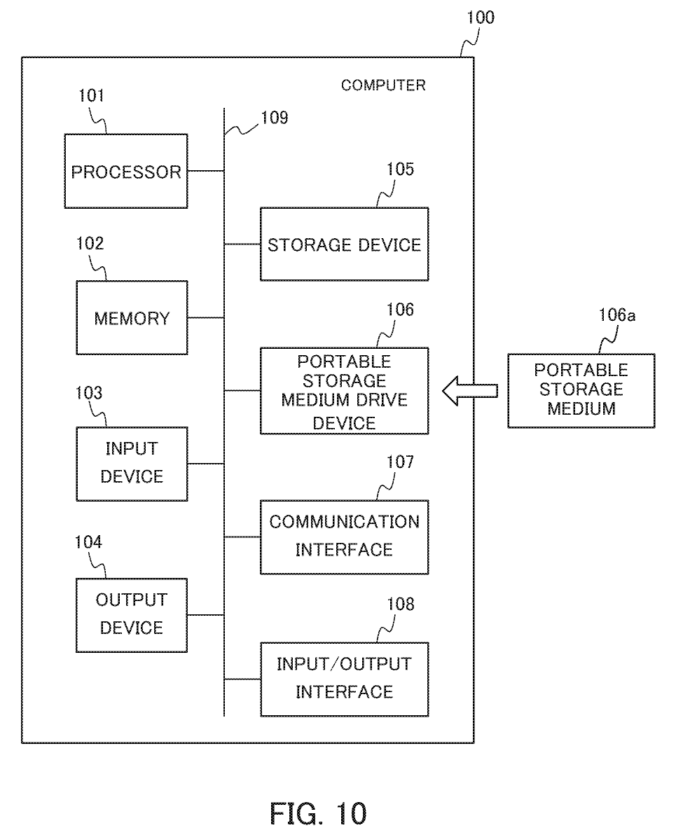

In addition, in the present embodiment, the function of a part (for example, the control unit 34 or the like) of the main body portion 30 may be implemented by an external control device, and the endoscopic device 1 may be implemented as an endoscopic system including the endoscopic device and the control device. In this case, the control device may be implemented by the computer 100 illustrated in FIG. 10.

FIG. 10 is a diagram illustrating a hardware configuration of a computer. The computer 100 illustrated in FIG. 10 includes a processor 101, a memory 102, an input device 103, an output device 104, a storage device 105, a portable storage medium drive device 106, a communication interface 107, and an input/output interface 108, and each of them is connected to a bus 109 and can transmit and receive data to and from one another.

The processor 101 is a CPU or the like, and performs various processing by executing a program such as an operating system (OS) or an application. The memory 102 includes RAM and ROM. A part of the program executed by the processor 101 or the like is temporarily stored in the RAM. In addition, the RAM is also used as a work area of the processor 101. The ROM stores a program executed by the processor 101, various data necessary for executing the program, and the like.

The input device 103 is a keyboard, a mouse, a touch panel, a joystick, or the like. The output device 104 is a display device such as an LCD.

The storage device 105 is a device that stores data, and is an HDD, an SSD, or the like. The portable storage medium drive device 106 drives a portable storage medium 106a, accesses the stored contents, and reads and writes data. The portable storage medium 106a is a memory device, a flexible disk, an optical disk, a magneto-optical disk, or the like. The portable storage medium 106a also includes compact disc read only memory (CD-ROM), a digital versatile disc (DVD), a Blu-ray disc, a USB memory, an SD card memory, and the like.

The communication interface 107 is an interface connected to a network in a wired or wireless manner and performing communication with the external device connected to the network. The input/output interface 108 is an interface connected to an external device such as an endoscopic device and inputs/outputs data to/from the external device.

In such a computer 100, the program executed by the processor 101 and various data necessary for executing the program are stored not only in the memory 102, and but may be stored in the storage device 105 or the portable storage medium 106a. In addition, the program executed by the processor 101 and various data necessary for executing the program may be stored in one or more of the memory 102, the storage device 105, and the portable storage medium 106a from an external device via the network and the communication interface 107.

In addition, the computer 100 is not limited to the one illustrated in FIG. 10, and may be configured to include a plurality of some components illustrated in FIG. 10 or may be configured without some components. For example, the computer 100 may include a plurality of processors.

In addition, the computer 100 may be configured to include hardware such as a microprocessor, a digital signal processor (DSP), an application specific integrated circuit (ASIC), a programmable logic device (PLD), or a field programmable gate array (FPGA). For example, the processor 101 may be implemented using at least one of these pieces of hardware.

Although the embodiment of the present invention has been described above, the present invention is not limited to the above-described embodiment, and various improvements and changes can be made without departing from the gist of the present invention.

What is claimed is:

1. A frame image extraction method comprising:
during recording processing of a moving image including a plurality of frame images generated on a basis of an imaging signal output from an imaging element included in an insertion portion inserted into a test subject, among the plurality of frame images, adding, as a tag, information regarding an operation to a frame image corresponding to a timing when an operation receiving unit receives the operation, and adding, as a tag, information regarding a specific feature image to a frame image recognized as the specific feature image; and
extracting a frame image from among the plurality of frame images included in the moving image on a basis of at least one type of tag among two or more types of tags selected from among a plurality of types of the tags.

2. The frame image extraction method according to claim 1, further comprising determining the specific feature image according to a feature image selection operation that has been received before the recording processing of the moving image.

3. The frame image extraction method according to claim 1, further comprising determining the specific feature image on a basis of a content of a tag related to the specific feature image that has been added to a frame image included in a moving image recorded by recording processing performed previously with respect to a test subject of a same type.

4. The frame image extraction method according to claim 1, further comprising obtaining the specific feature image by machine learning of a feature of a frame image to which a specific type of a tag is added, included in a moving image recorded by recording processing performed previously with respect to a test subject of a same type.

5. The frame image extraction method according to claim 1, further comprising selecting the two or more types of tags according to a tag selection operation that has been received.

6. The frame image extraction method according to claim 5, further comprising displaying tags added to a frame image included in the moving image,
wherein the tag selection operation is an operation of selecting the two or more types of tags from the tags that have been displayed.

7. The frame image extraction method according to claim 6, further comprising displaying a time bar of the moving image and identifiably displaying a tag added to a frame image included in the moving image on the time bar.

8. The frame image extraction method according to claim 5, further comprising displaying a time bar of the moving image and identifiably displaying a tag added to a frame image included in the moving image on the time bar,
wherein the tag selection operation is an operation of selecting the two or more types of tags by region designation from among the tags identifiably displayed on the time bar that have been displayed.

9. The frame image extraction method according to claim 1, further comprising:
selecting some types of tags among the two or more types of tags in advance, and
selecting an other type of tag according to a tag selection operation that has been received.

10. The frame image extraction method according to claim 1, further comprising extracting a corresponding section in the moving image for each of the two or more types of tags, and performing the extraction on a basis of the section.

11. The frame image extraction method according to claim 1, further comprising:
displaying a frame image that has been extracted.
